(19) United States
(12) Patent Application Publication
(10) Pub. No.: US 2003/0120177 A1
(43) Pub. Date: Jun. 26, 2003

(54) Blood-collecting tube setting system

(76) Inventor: Teruaki Itoh, Kumamoto-shi (JP)

Correspondence Address: Nixon & Vanderhye P.C., 8th Floor, 1100 North Glebe Road., Arlington, VA, 22201-4714, US

(21) Appl. No.: 10/294,552

(22) Filed: Nov. 15, 2002

(30) Foreign Application Priority Data
Nov. 28, 2001 (JP) 2001-362816

(51) Int. Cl. A61B 5/00

(52) U.S. Cl. 600/573

(57) ABSTRACT

A blood-collecting tube setting system includes an information collecting unit which collects information of a blood donor accepted at a reception desk, a sheet-issuing unit which issues a pair of identification sheets having the same recording contents corresponding to the collected information, a sheet-sticking unit which sticks one of the issued identification sheets on an unused blood-collecting tube, a sheet-attaching member which attaches the other of the issued identification sheets to the blood donor, a first reading unit which reads an information item of a blood-collecting tube identification sheet, a second reading unit which reads an information item of a blood donor identification sheet, a comparison/determination unit which compares the read two information items and determines whether both the information items are matched or not, and an informing unit which informs an operator of results of comparison and determination.

### CROSS-REFERENCE TO RELATED APPLICATIONS

This application is based upon and claims the benefit of priority from the prior Japanese Patent Application No. 2001-362816, filed Nov. 28, 2001, the entire contents of which are incorporated herein by reference.

### BACKGROUND OF THE INVENTION

1. Field of the Invention

The present invention relates to a blood-collecting tube setting system for preparing blood-collecting tubes for collecting blood from a blood donor in a hospital and the like.

2. Description of the Related Art

For example, Japanese Patent No. 2739928 discloses a blood-collecting tube preparing system as a prior art blood-collecting tube setting system. In the system disclosed in the Japanese Patent, a reception area (room) and a blood-collecting area (room) are separated from each other.

In the reception area, an operator collects information of a blood donor, records the collected information on a label, sticks the label on a blood-collecting tube, carries out the labeled blood-collecting tube to the blood-collecting area, and the like. In the blood-collecting area, an operator reads information from the label stuck on the carried blood-collecting tube, displays the read information, calls the blood donor who stands by in the reception area, collects blood of the blood donor, and the like.

According to the above prior art blood-collecting tube preparing system, an operator calls blood donors in sequence into the blood-collecting area and collects blood of the donors based on the information of the labels stuck on the blood-collecting tubes carried into the blood-collecting area. In this case, the operator confirms a correlation between a labeled blood-collecting tube and a called blood donor by his or her own matching operation. Thus, there is a fear that an error will be caused in the correlation by human error.

### BRIEF SUMMARY OF THE INVENTION

An object of the present invention is to provide a blood-collecting tube setting system having the following advantages.

1. It is possible to prevent a check error due to a mismatch between a blood-collecting tube on which an identification sheet is stuck and a called blood donor.

2. The configuration of the system is easy to achieve.

In order to attain the above object, the blood-collecting tube setting system according to the present invention has the following characteristic configuration. The other characteristic configurations will be clarified in the Embodiments later.

A blood-collecting tube setting system according to the present invention, comprises an information collecting unit which collects information of a blood donor accepted at a reception desk, a sheet-issuing unit which issues a pair of identification sheets having same recording contents corresponding to the information collected by the information collecting unit, a sheet-sticking unit which sticks one of the identification sheets issued by the sheet-issuing unit on an unused blood-collecting tube, sheet-attaching means for attaching the other of the identification sheets issued by the sheet-issuing unit to the blood donor, a first reading unit which reads an information item of a blood-collecting tube identification sheet stuck on the unused blood-collecting tube, a second reading unit which reads an information item of a blood donor identification sheet attached to the blood donor, a comparison/determination unit which compares the information item read by the first reading unit and the information item read by the second reading unit and determines whether both the information items are matched or not, and an informing unit which informs an operator of results of comparison and determination of the comparison/determination unit.

In the blood-collecting tube setting system described above, a correlation between a blood-collecting tube and a blood donor is automatically confirmed by reading and comparing an information item of a blood-collecting tube identification sheet stuck on the blood-collecting tube and an information item of the blood donor identification sheet attached to the blood donor. Consequently, there is no fear that an error will be caused in the correlation by human error, and a check error due to a mismatch in the correlation can be prevented. Furthermore, the configuration of the system is simple and easy to achieve.

### BRIEF DESCRIPTION OF THE SEVERAL VIEWS OF THE DRAWING

FIG. 1 is a block diagram showing a configuration of a blood-collecting tube setting system according to a first embodiment of the present invention;

FIG. 2 is a side view of a blood-collecting tube on which a blood-collecting tube identification sheet is stuck in the system according to the first embodiment of the present invention;

FIG. 3A is a front view of an attaching member on which a blood donor identification sheet is stuck in the system according to the first embodiment of the present invention;

FIG. 3B is a side view of the attaching member shown in FIG. 3A;

FIG. 4 is a block diagram showing a configuration of a blood-collecting tube setting system according to a second embodiment of the present invention; and

FIG. 5 is a block diagram showing a configuration of a blood-collecting tube setting system according to a third embodiment of the present invention.

### DETAILED DESCRIPTION OF THE INVENTION

(First Embodiment)

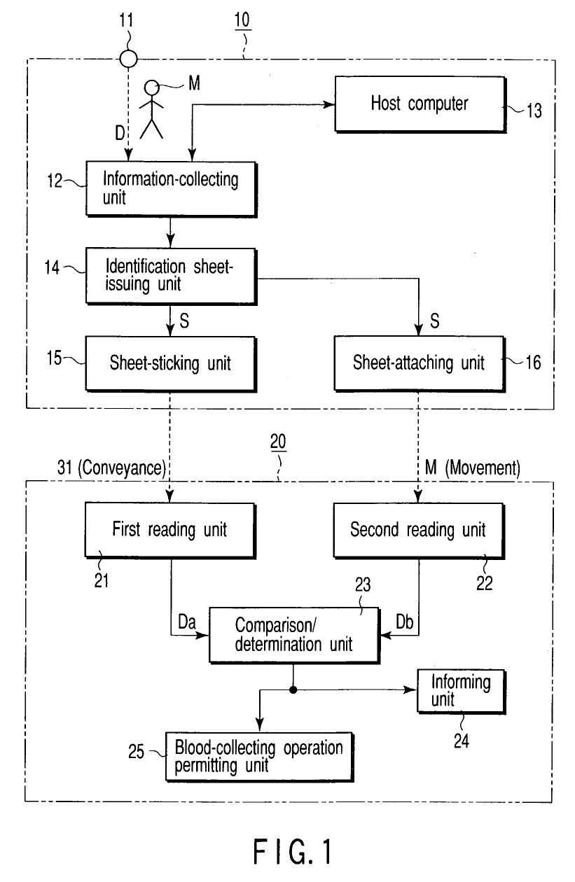

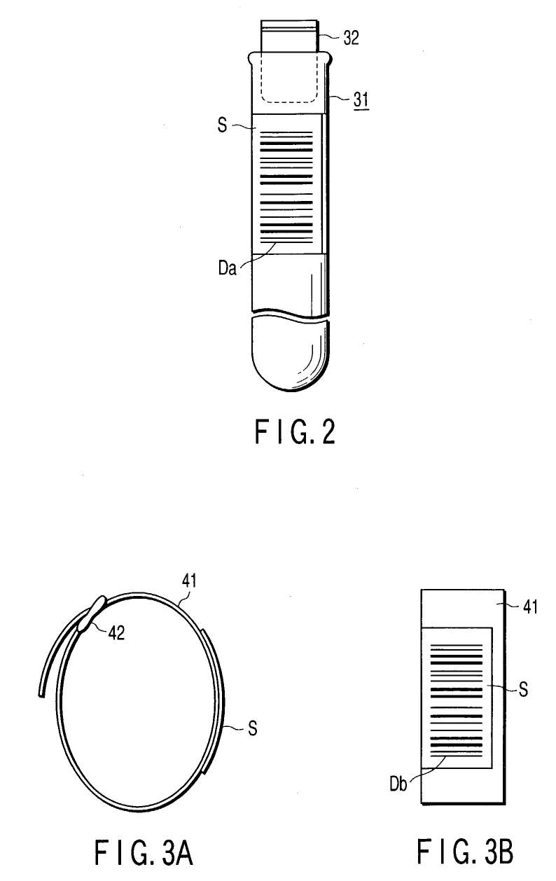

In FIG. 1, reference numeral 10 indicates a reception area (room) and reference numeral 20 denotes a blood-collecting area (room).

The reception area 10 includes an information-collecting unit 12 that collects information D of a blood donor M accepted at a reception desk 11. The unit 12 collects the information D of the blood donor M by communicating with a host computer 13. An identification sheet-issuing unit 14 converts the information D collected by the unit 12 into, e.g., a bar code, records it on a label and issues a pair of identification sheets S having the same recording contents at the same time.

A sheet-sticking unit (e.g., an automatic label sticking unit) 15 sticks one of the identification sheets S issued at the same time on the outer-surface of an unused blood-collecting tube 31. The opening of the unused blood-collecting tube 31 is stopped with a stopper 32.

A sheet-attaching means 16 attaches the other issued identification sheet S to the blood donor M.

As shown in FIGS. 3A and 3B, the sheet-attaching means 16 allows the identification sheet S to be attached to the blood donor M by fixing the identification sheet S to one side of a wristband type attaching member (e.g., band-like resin, Kraft paper, nonwoven fabric, ordinary paper, leather and cotton cloth) 41 using a fixing means (e.g., an adhesion means, a pin and an insertion means).

The attaching member 41 has a watchband-shaped stopper 42. Therefore, the attaching member 41 has to be wound on a wrist of the blood donor M by turning off the stopper 42 and then fixed on the wrist by turning on the stopper 42.

Returning to FIG. 1, the unused blood-collecting tube 31 on which the identification sheet S is stuck is automatically conveyed to the blood-collecting area 20 by, for example, a conveyor belt type conveying mechanism (not shown). The blood donor M with the identification sheet S on part of the body thereof is called by announcement and moved to the blood-collecting area 20.

The blood-collecting area 20 includes a first reading unit 21 and a second reading unit 22. The first reading unit 21 reads an information item Da of the blood-collecting tube identification sheet S stuck on the blood-collecting tube 31 conveyed from the reception area 10. The second reading unit 22 reads an information item Db of the blood donor identification sheet S attack on the blood donor M who is moved from the reception area 10.

A comparison/determination unit 23 compares the information item Da read by the first reading unit 21 and the information item Db read by the second reading unit 22 and determines whether the information items Da and Db are matched or not. An informing unit 24 including a display device and a speech generation device informs an operator of the results of the determination or the contents as to whether both the information items are matched or not.

When the comparison/determination unit 23 determines that both the information items are matched with each other, a blood-collecting operation permitting unit 25 causes, for example, a robot arm (not shown) to move the blood-collecting tube 31 to its usable position to permit a blood-collecting operator to collect blood.

The blood-collecting operator performs or stops the blood-collecting operation based on the determination results obtained by the informing unit 24. When both the information items are matched with each other, the blood collection of the blood donor M is performed using the blood-collecting tube 31 that is moved to its usable position.

When both the information items are not matched with each other for some reason, the blood-collecting operation permitting unit 25 does not permit a blood-collecting operation. In other words, the blood-collecting tube 31 does not move to its usable position. Thus, the blood-collecting operator cannot perform the blood-collecting operation even though he or she misidentifies the determination result from the informing unit 24. It is therefore unlikely that a check error will occur, and the system of the present invention is very safe.

(Second Embodiment)

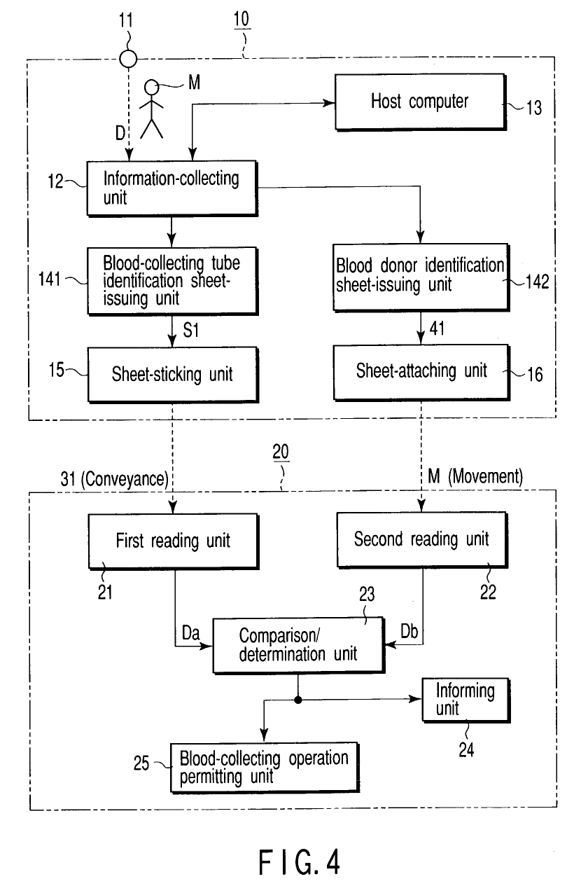

FIG. 4 is a block diagram showing a configuration of a blood-collecting tube setting system according to a second embodiment of the present invention. The second embodiment differs from the first embodiment in that a blood-collecting tube identification sheet-issuing unit 141 for issuing a blood-collecting tube identification sheet S1 and a blood donor identification sheet-issuing unit 142 for issuing a blood donor identification sheet S2 are provided independently of each other instead of using the identification sheet-issuing unit 14.

In the system of the second embodiment, the blood donor identification sheet S2 can be issued in its own fashion other than that of the blood-collecting tube identification sheet S1. The second embodiment therefore has the advantage that the blood donor identification sheet S2, which is attached to a blood donor M, can be issued in the most favorable fashion that matches the conditions of use and the like. Since the other features are the same as those of the first embodiment, their descriptions are omitted.

(Third Embodiment)

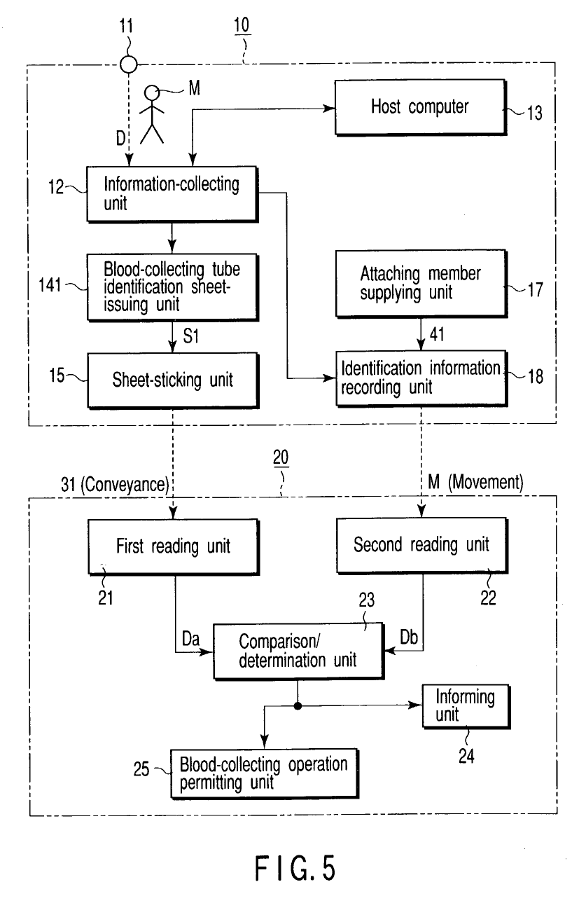

FIG. 5 is a block diagram showing a configuration of a blood-collecting tube setting system according to a third embodiment of the present invention. The third embodiment differs from the second embodiment in that an attaching member supplying unit 17 supplies a predetermined member such as the above wristband type attaching member 41 and an identification information recording unit 18 directly records identification information on the attaching member 41. Printing, punching, magnetic, electrostatic, and magneto-optic recording units are appropriately used as the identification information recording unit 18. It is preferable that the attaching member 41 be obtained by forming a recording area that matches the above recording unit in part of the above-described band-like resin, Kraft paper, nonwoven fabric, ordinary paper, leather and cotton cloth.

In the system of the third embodiment, an identification information item Db is directly recorded on the attaching member 41. An operator can thus save trouble in sticking the identification sheet S after he or she creates it. Since the other features are the same as those of the second embodiment, their descriptions are omitted.

(Features of the Embodiments)

[1] A blood-collecting tube setting system according to an embodiment, comprises:

an information-collecting unit 12 which collects information D of a blood donor M accepted at a reception desk 11;

a sheet-issuing unit 14 which issues a pair of identification sheets S having the same recording contents corresponding to the information D collected by the information-collecting unit 12;

a sheet-sticking unit (e.g., an automatic label sticking unit) 15 which sticks one of the identification sheets S issued by the sheet-issuing unit 14 on an unused blood-collecting tube 31;

sheet-attaching means (e.g., wristband type attaching means and suspended cord type attaching means) 16 for attaching the other of the identification sheets S issued by the sheet-issuing unit 14 to the blood donor M;

a first reading unit 21 which reads an information item Da of a blood-collecting tube identification sheet S1 stuck on the unused blood-collecting tube 31;

a second reading unit 22 which reads an information item Db of a blood donor identification sheet S2 attached to the blood donor M;

a comparison/determination unit 23 which compares the information item Da read by the first reading unit 21 and the information item Db read by the second reading unit 22 and determines whether both the information items Da and Db are matched or not; and

an informing unit (e.g., a display device and a speech generation device) 24 which informs an operator of results of comparison and determination of the comparison/determination unit 23.

In the blood-collecting tube setting system described above, a correlation between the blood-collecting tube 31 and the blood donor M is automatically confirmed by reading and comparing the information item Da of the blood-collecting tube identification sheet S1 stuck on the blood-collecting tube 31 and the information item Db of the blood donor identification sheet S2 attached to the blood donor M. Consequently, there is no fear that an error will be caused in the correlation by human error, and a check error due to a mismatch in the correlation can be prevented. Furthermore, the configuration of the system is simple and easy to achieve.

[2] A blood-collecting tube setting system according to an embodiment, comprises:

an information-collecting unit 12 which collects information D of a blood donor M accepted at a reception desk 11;

a blood-collecting tube identification sheet-issuing unit 141 which issues a blood-collecting tube identification sheet S1 on which the information D collected by the information-collecting unit 12 is recorded;

a sheet-sticking unit (e.g., an automatic label sticking unit) 15 which sticks the blood-collecting tube identification sheet S1 issued by the blood-collecting tube identification sheet-issuing unit 141 on an unused blood-collecting tube 31;

a blood donor identification sheet-issuing unit 142 which issues a blood donor identification sheet S2 on which the information D collected by the information collecting unit 12 is recorded;

sheet-attaching means (e.g., wristband type attaching means and suspended cord type attaching means) 16 for attaching the blood donor identification sheet S2 issued by the blood donor identification sheet-issuing unit 142 to the blood donor M;

a first reading unit 21 which reads an information item Da of the blood-collecting tube identification sheet S1 stuck on the unused blood-collecting tube 31;

a second reading unit 22 which reads an information item Db of the blood donor identification sheet S2 attached to the blood donor M;

a comparison/determination unit 23 which compares the information item Da read by the first reading unit 21 and the information item Db read by the second reading unit 22 and determines whether both the information items Da and Db are matched or not; and

an informing unit (e.g., a display device and a speech generation device) 24 which informs an operator of results of comparison and determination of the comparison/determination unit 23.

In the blood-collecting tube setting system described above, the blood donor identification sheet S2 is issued in its own fashion other than that of the blood-collecting tube identification sheet S1. The blood donor identification sheet S2 can be issued in the most favorable fashion when it is attached to the blood donor M in accordance with the conditions of use and the like.

[3] A blood-collecting tube setting system according to an embodiment, comprises:

an information-collecting unit 12 which collects information D of a blood donor M accepted at a reception desk 11;

a blood-collecting tube identification sheet-issuing unit 141 which issues a blood-collecting tube identification sheet S1 on which the information D collected by the information collecting unit 12 is recorded;

a sheet-sticking unit (an automatic label sticking unit) 15 which sticks the blood-collecting tube identification sheet SI issued by the blood-collecting tube identification sheet-issuing unit 141 on an unused blood-collecting tube 31;

an attaching member supplying unit 17 which supplies an attaching member 41 that is attached to the blood donor M;

an identification information recording unit 18 (e.g., printing, punching, magnetic, electrostatic, and magneto-optic recording units) which records the information D collected by the information collecting unit 12 on the attaching member 41 supplied by the attaching member supplying unit 17, as an identification information item Db;

a first reading unit 21 which reads an information item Da of the blood-collecting tube identification sheet S1 stuck on the unused blood-collecting tube 31;

a second reading unit 22 which reads the identification information item Db recorded on the attaching member 41 attached to the blood donor M;

a comparison/determination unit 23 which compares the information item Da read by the first reading unit 21 and the identification information item Db read by the second reading unit 22 and determines whether both the information items Da and Db are matched or not; and

an informing unit (e.g., a display device and a speech generation device) 24 which informs an operator of results of comparison and determination of the comparison/determination unit 23.

The foregoing blood-collecting tube setting system has the advantage that since the information D is directly recorded on the attaching member 41, no additional identification sheets S have to be prepared and thus the process of manufacturing the system can be simplified.

[4] The blood-collecting tube setting system according to the embodiment, described in one of the above paragraphs [1] to [3], further comprises a blood-collecting operation permitting unit 25 which permits a blood-collecting operation (e.g., a movement of the blood-collecting tube 31 to its usable position) using the blood-collecting tube 31 when the comparison/determination unit 23 determines that the information items Da and Db read by the first and second reading units 21 and 22 are matched with each other.

In the blood-collecting tube setting system described above, no permission to perform a blood-collecting operation is given unless the information items Da and Db are matched with each other. If, therefore, a blood-collecting operator misidentifies the determination result of the comparison/determination unit 23 obtained from the informing unit 24, the blood-collecting operation is inhibited. It is therefore possible to prevent a check error due to a mismatch in the above-described correlation more reliably.

[5] In the blood-collecting tube setting system according to the embodiment, described in one of the above paragraphs [1] and [2], the sheet-attaching means 16 allows the identification sheet S to be attached to the blood donor M by fixing the identification sheet S to an attaching member (e.g., a wristband with a stopper and a cord dangled from one's neck) 41 using a fixing member (e.g., an adhesion means, a pin and an insertion means).

[6] In the blood-collecting tube setting system according to the embodiment, described in one of the above paragraphs [1] to [5], the information D is recorded in barcode recording mode.

The foregoing blood-collecting tube setting system can be configured at low cost with high reliability since the information D is recorded in barcode recording mode for general purpose use.

(Modifications)

The blood-collecting tube setting system according to each of the embodiments contains the following modifications:

The system can comprise sheet-attaching means having a clip attached to part of a transparent case capable of holding an identification sheet, which catches a given portion of user's clothes by the clip.

The system can comprise sheet-attaching means having an insertion clip attached to part of a transparent case capable of holding an identification sheet, which attaches the identification sheet by inserting the insertion pin into a given portion of user's clothes.

Information is recorded in recording mode other than the barcode recording mode.

What is claimed is:

1. A blood-collecting tube setting system comprising:
an information-collecting unit which collects information of a blood donor accepted at a reception desk;
a sheet-issuing unit which issues a pair of identification sheets having same recording contents corresponding to the information collected by the information collecting unit;
a sheet-sticking unit which sticks one of the identification sheets issued by the sheet-issuing unit on an unused blood-collecting tube;
sheet-attaching means for attaching the other of the identification sheets issued by the sheet-issuing unit to the blood donor;
a first reading unit which reads an information item of a blood-collecting tube identification sheet stuck on the unused blood-collecting tube;
a second reading unit which reads an information item of a blood donor identification sheet attached to the blood donor;
a comparison/determination unit which compares the information item read by the first reading unit and the information item read by the second reading unit and determines whether both the information items are matched or not; and
an informing unit which informs an operator of results of comparison and determination of the comparison/determination unit.

2. A blood-collecting tube setting system comprising:
an information-collecting unit which collects information of a blood donor accepted at a reception desk;
a blood-collecting tube identification sheet-issuing unit which issues a blood-collecting tube identification sheet on which the information collected by the information-collecting unit is recorded;
a sheet-sticking unit which sticks the blood-collecting tube identification sheet issued by the blood-collecting tube identification sheet-issuing unit on an unused blood-collecting tube;
a blood donor identification sheet-issuing unit which issues a blood donor identification sheet on which the information collected by the information-collecting unit is recorded;
sheet-attaching means for attaching the blood donor identification sheet issued by the blood donor identification sheet-issuing unit to the blood donor;
a first reading unit which reads an information item of the blood-collecting tube identification sheet stuck on the unused blood-collecting tube;
a second reading unit which reads an information item of the blood donor identification sheet attached to the blood donor;
a comparison/determination unit which compares the information item read by the first reading unit and the information item read by the second reading unit and determines whether both the information items are matched or not; and
an informing unit which informs an operator of results of comparison and determination of the comparison/determination unit.

3. A blood-collecting tube setting system comprising:
an information-collecting unit which collects information of a blood donor accepted at a reception desk;
a blood-collecting tube identification sheet-issuing unit which issues a blood-collecting tube identification sheet on which the information collected by the information-collecting unit is recorded;
a sheet-sticking unit which sticks the blood-collecting tube identification sheet issued by the blood-collecting tube identification sheet-issuing unit on an unused blood-collecting tube;
an attaching member supplying unit which supplies an attaching member that is attached to the blood donor;
an identification information recording unit which records the information collected by the information collecting unit on the attaching member supplied by the attaching member supplying unit, as an identification information item;
a first reading unit which reads an information item of the blood-collecting tube identification sheet stuck on the unused blood-collecting tube;
a second reading unit which reads the identification information item recorded on the attaching member attached to the blood donor;
a comparison/determination unit which compares the information item read by the first reading unit and the identification information item read by the second reading unit and determines whether both the information items are matched or not; and
an informing unit which informs an operator of results of comparison and determination of the comparison/determination unit.

4. The blood-collecting tube setting system according to claim 1, further comprising a blood-collecting operation permitting unit which permits a blood-collecting operation using the blood-collecting tube when the comparison/determination unit determines that the information items read by the first and second reading units are matched with each other.

5. The blood-collecting tube setting system according to claim 2, further comprising a blood-collecting operation permitting unit which permits a blood-collecting operation using the blood-collecting tube when the comparison/determination unit determines that the information items read by the first and second reading units are matched with each other.

6. The blood-collecting tube setting system according to claim 3, further comprising a blood-collecting operation permitting unit which permits a blood-collecting operation using the blood-collecting tube when the comparison/determination unit determines that the information items read by the first and second reading units are matched with each other.

7. The blood-collecting tube setting system according to claim 1, wherein the sheet-attaching means allows the identification sheet to be attached to the blood donor by fixing the identification sheet to an attaching member using a fixing member.

8. The blood-collecting tube setting system according to claim 2, wherein the sheet-attaching means allows the identification sheet to be attached to the blood donor by fixing the identification sheet to an attaching member using a fixing member.

9. The blood-collecting tube setting system according to claim 1, wherein the information is recorded in barcode recording mode.

10. The blood-collecting tube setting system according to claim 2, wherein the information is recorded in barcode recording mode.

11. The blood-collecting tube setting system according to claim 3, wherein the information is recorded in barcode recording mode.

12. The blood-collecting tube setting system according to claim 4, wherein the information is recorded in barcode recording mode.

13. The blood-collecting tube setting system according to claim 5, wherein the information is recorded in barcode recording mode.

14. The blood-collecting tube setting system according to claim 6, wherein the information is recorded in barcode recording mode.

15. The blood-collecting tube setting system according to claim 7, wherein the information is recorded in barcode recording mode.

16. The blood-collecting tube setting system according to claim 8, wherein the information is recorded in barcode recording mode.
